(19) United States
(12) Patent Application Publication
(10) Pub. No.: US 2006/0246123 A1
(43) Pub. Date: Nov. 2, 2006

(54) Oligonucleotide mimetics

(76) Inventors: Eli Gilboa, Durham, NC (US); Sandra L. Santulli-Marotto, Carrboro, NC (US); Bruce A. Sullenger, Durham, NC (US); Christopher P. Rusconi, Durham, NC (US)

Correspondence Address: NIXON & VANDERHYE, PC, 901 NORTH GLEBE ROAD, 11TH FLOOR, ARLINGTON, VA, 22203, US

(21) Appl. No.: 10/548,532

(22) PCT Filed: Mar. 12, 2004
(86) PCT No.: PCT/US04/07405

§ 371 Date: May 15, 2006

Related U.S. Application Data
(60) Provisional application No. 60/453,831, filed on Mar. 12, 2003.

(51) Int. Cl. A61K 48/00; C12Q 1/68; A61K 9/127

(52) U.S. Cl. 424/450; 514/44; 435/6

(57) ABSTRACT

The present invention relates, in general, to a method of using aptamers to modulate the immune system and, in method of inhibiting CTLR-4 function and to aptamers suitable for use in such a method.

This application claims priority from Prov. Appln. No. 60/453,831, filed Mar. 12, 2003, the entire contents of that application being incorporated herein by reference.

### TECHNICAL FIELD

The present invention relates, in general, to a method of using aptamers to modulate the immune system and, in particular, to a method of inhibiting CTLR-4 function and to aptamers suitable for use in such a method.

### BACKGROUND

A critical event in T cell activation is the interaction between the T cell receptor (TCR) and the MHC-peptide complex on the antigen presenting cell (APC). In the mid 80s it became clear that a second costimulatory signal is necessary for full activation. CD28 is the major costimulatory molecule expressed on the surface of resting as well as activated T cells and B7-1 and B7-2 are the main counterreceptors of CD28 expressed on professional APC, such as dendritic cells and activated monocyte/macrophages (Chambers et al, Immunity 7(6):885-895 (1997), Alegre et al, Nature Reviews Immunology 1:220-228 (2001), Salomon and Bluestone, Annu. Rev. Immunol. 19:225-252 (2001)). In the T cell activation process, signaling via CD28 (costimulation) is necessary for IL-2 production by regulating transcription and stability of IL-2 mRNA. Although a potent costimulator of T cell activation, CD28 function is not always required, especially under circumstances where a strong or sustained antigen (Ag)-specific signal is available (Teh and Teh, Cell Immunol. 179(1):74-83 (1997), Kundig et al, Immunity 5(1):41-52 (1996)).

CTLA-4 is a second costimulatory molecule which shares considerable homology with CD28, including a motif, MYPPY, involved in binding to their common ligands, B7-1 and B7-2 (Linsley et al, Immunity 1(9):793-801 (1994)). Unlike CD28, expression of CTLA-4 is induced upon T cell activation and CTLA-4 binds to the B7 ligands with 50-2000 higher affinity than CD28 (Brunet et al, Nature 328(6127):267-270 (1987)). CTLA-4 is a negative regulator of T cell activation (reviewed in Alegre et al, Nature Reviews Immunology 1:220-228 (2001), Salomon and Bluestone, Annu. Rev. Immunol. 19:225-252 (2001), Chambers et al, Immunity 7(6):885-895 (1997), Egen et al, Nat. Immunol. 3(7):611-618 (2002)). Initial evidence came from in vitro studies showing that inhibition of CTLA-4 mediated signaling with monovalent anti-CTLA-4 antibody (Ab) enhanced Ag-dependent (and CD28 dependent) T cell proliferation (Krummel and Allison, J. Exp. Med. 182(2):459-465 (1995), Walunas et al, Immunity 1(5):405-413 (1994)). Compelling evidence of the inhibitory function of CTLA-4 came from observations that CTLA-4 deficient mice developed a fatal lymphoproliferative disorder (Waterhouse et al, Science 270(5238):985-988 (1995), Tivol et al, Immunity 3(5):541-547 (1995)). Whereas CTLA-4 is activated on both CD4+ and CD8+ T cells, it appears that in vivo CD4+ T cells are the primary targets for CD4 T cell mediated inhibition (Chambers et al, Immunity 7(6):885-895 (1997), Bachmann et al, J. Immunol. 160(1):95-100 (1998)). CD4+CD25+ regulatory T cells (Treg) express constitutively CTLA-4 (Read et al, J. Exp. Med. 192(2):295-302 (2000), Takahashi et al, J. Exp. Med. 192(2):303-310 (2000)) but the functional role of CTLA-4 on Treg is at present controversial (Read et al, J. Exp. Med. 192(2):295-302 (2000), Takahashi et al, J. Exp. Med. 192(2):303-310 (2000), Levings et al, J. Exp. Med. 193(11):1295-1302 (2001), Jonuleit et al, J. Exp. Med. 193(11):1285-1294 (2001)). Since the genetic absence of CTLA-4 in knockout mice (Waterhouse et al, Science 270(5238):985-988 (1995), Tivol et al, Immunity 3(5):541-547 (1995)) or Ab mediated blockade of CTLA-4 function in normal mice (Takahashi et al, J. Exp. Med. 192(2):303-310 (2000)) is associated with massive lymphoproliferation, it is conceivable that the physiological function of CTLA-4 is to maintain peripheral tolerance, namely prevent the activation and/or attenuate the expansion of autoreactive T cells. Whether this is mediated by CTLA-4 expressed on the autoreactive T cells or on Treg cells, or a combination of both, is not clear.

Given the inhibitory role of CTLA-4 on T cell activation manifested by the ability of CTLA-4 blockade to enhance T cell responses in vitro and the extensive T cell proliferation seen in CTLA-4 deficient mice, it was reasonable to test whether transient inhibition of CTLA-4 function in vivo is capable of enhancing tumor immunity. Several studies using murine tumor models have indeed shown that blockade of CTLA-4 in vivo enhanced antitumor T-cell lo dependent immunity, providing further evidence for a role of CTLA-4 in attenuating Ag-specific polyclonal T cell responses. In these studies, transient CTL4 blockade was achieved by treating mice with an anti-CTL4 monoclonal Ab.

It was initially shown that treatment of mice with anti-CTLA-4 Ab leads to the rejection of immunogenic transplanted tumors but had no or little effect on weakly or non immunogenic tumors (Yang et al, Cancer Res. 57(18):4036-4041 (1997), Leach et al, Science 271(5256):1734-1736 (1996)). Rejection of non immunogenic tumors, including preestablished tumors, could be achieved if CTLA-4 blockade were used in combination with vaccination (Hurwitz et al, Proc. Natl. Acad. Sci. USA 95(17):10067-10071 (1998), Hurwitz et al, Cancer Res. 60(9):2444-2448 (2000), van Elsas et al, J. Exp. Med. 190(3):355-366 (1999)) or low dose chemotherapy (Mokyr et al, Cancer Res. 58(23):5301-5304 (1998)), under condition such that neither treatment was effective alone. These observations reinforce the view that CTLA-4 blockade in vivo facilitates the Ag dependent activation and/or expansion of T cells by blocking inhibitory signals delivered by CTLA-4. Interestingly, a recent study has shown that concomitant depletion of both CD4+CD25+ regulatory T cells (using anti-CD25 Ab) and CTLA-4 blockade (using anti-CTLA-4 Ab) had a synergistic antitumor effect, suggesting that CTLA-4 mediated immunosuppression is mediated by a pathway and cells which are different from the CD4+CD25+ regulatory T cells (Sutmuller et al, J. Exp. Med. 194(6):823-832 (2001)). In some instances, treatment of mice with anti-CTLA-4 Ab combined with vaccination was associated with mild autoimmune manifestations in the form of local skin depigmentation (Hurwitz et al, Cancer Res. 60(9):2444-2448 (2000), van Elsas et al, J. Exp. Med. 190(3):355-366 (1999), Sutmuller et al, J. Exp. Med. 194(6):823-832 (2001), van Elsas et al, J. Exp. Med. 194(4):481-489 (2001)). This may represent the activation of autoreactive T cells directed against tissue-specific antigens expressed in the tumor vaccine, not unlike what is seen in CTLA-4 deficient mice (Waterhouse et al, Science 270(5238):985-988 (1995), Tivol et al, Immunity 3(5):541-547 (1995)).

Aptamers are high affinity single stranded nucleic acid ligands, each specific for a given target molecule, that can be isolated through a combinatorial chemistry process using iterative in vitro selection techniques. An approach to such in vitro selection is outlined in FIG. 1 (designated SELEX (systematic evolution of ligands by exponential enrichment) (Ellington and Szostak, Nature 346(6287):818-822 (1990), Tuerk and Gold, Science 249(4968):505-510 (1990)). SELEX is a powerful purification method in which very rare binding activities (with frequencies of 1 in 10¹¹ to 1 in 10¹³) can be isolated by affinity purification from a large combinatorial library. The starting point for the in vitro selection process is a combinatorial library composed of single-stranded nucleic acids (RNA, DNA, or modified RNA) usually containing 20-40 randomized positions. Randomization creates an enormous diversity of possible sequences (e.g., four different nucleotides at 40 randomized positions give a theoretical possibility of 4⁴⁰ or 10²⁴ different sequences). Because short single-stranded nucleic acids adopt fairly rigid structures that are dictated by their sequences, such a library contains a vast number of molecular shapes or conformations. To isolate high affinity nucleic acid ligands to a given target protein, the starting library of nucleic acids (in practice 10¹⁴ to 10¹⁵ different sequences) is incubated with the protein of interest. Nucleic acid molecules that adopt conformations that allow them to bind to a specific protein are then partitioned from other sequences in the library that are unable to bind to the protein under the conditions employed. The bound sequences are then removed from the protein and amplified by reverse transcription and PCR (for RNA-based libraries) or just PCR (for DNA-based libraries) to generate a reduced complexity library enriched in sequences that bind to the target protein. This library is then transcribed in vitro (for RNA-based libraries), or its strands are separated (for DNA libraries) to generate molecules for use in the next round of selection. After several rounds (usually 8-12), which are typically performed with increasing stringency, the selected ligands are sequenced and evaluated for their affinity for the targeted protein and their ability to inhibit the activity of the targeted protein in vitro.

A SELEX isolated aptamer can exhibit remarkable affinity and specificity. If successfully performed, the selected ligands usually bind tightly with typical dissociation constants ranging from low picomolar (1×10⁻¹² M) to low nanomolar (1×10⁻⁹ M). As in vitro selection techniques have improved, the generation of aptamers with subnanomolar affinities for the target has become increasingly common. These affinities are similar to those measured for interactions between monoclonal antibodies and antigens. However, since the dissociation constants measured for aptamer-target proteins are true affinities, reflecting a bimolecular interaction in solution, they are more accurately compared to the affinities of Fab fragments for their target antigens. On average, the affinities of aptamers for a targeted protein are stronger than is typical for interactions between Fab fragments and their target antigens (Gold et al, Annu. Rev. Biochem. 64:763-797 (1995)). High-affinity nucleic acid-protein interactions require specific complementary contacts between functional groups on both the nucleic acid and the protein. Because the specific three-dimensional arrangement of complementary contact sites that mediate the protein-aptamer interaction are unlikely to be recapitulated in other proteins, aptamers are generally specific for their targets. By “toggling” the selection rounds between two related targets (such human and porcine thrombin (White et al, Mol. Ther. 4(6):567-573 (2001)), a crossreactive aptamer that binds to common motifs of the related targets can be isolated.

The present invention provides a method of modulating immune function using aptamers and to aptamers suitable for use in such a method. The aptamers of the invention can serve as a useful adjunct to, for example, Ag-specific immunotherapy.

### SUMMARY OF THE INVENTION

The present invention relates generally to a method of modulating the immune system using aptamers. More specifically, the invention relates to a method of inhibiting CTLA-4 function and to aptamers suitable for use in such a method.

Objects and advantages of the present invention will be clear from the description that follows.

### BRIEF DESCRIPTION OF THE DRAWINGS

FIG. 1: Schematic diagram of the SELEX protocol.

FIG. 2: Schematic diagram of positive-negative SELEX for CTLA-4. The RNA library is initially incubated with CD28 and RNAs that bind this protein are discarded. The precleared library is then incubated with CTLA-4 and RNAs that bind it are selected and amplified. This process is repeated for each round of selection until high affinity aptamers that distinguish between CTLA-4 and CD28 are isolated.

FIG. 3: Schematic diagram for TOGGLE SELEX against human and murine CTLA-4. In round #1, the RNA library is incubated with both murine and human CTLA-4 and RNA ligands are isolated and amplified that can bind either protein. In subsequent “even” rounds of selection, the library is incubated with human CTLA-4 and in “odd” rounds incubated with murine CTLA-4 to isolate aptamers that can bind to a conserved region present on both proteins.

FIGS. 4A and 4B. In vitro functional analysis of CTLA-4 binding aptamers. FIG. 4A. Inhibition of CTLA-4 functions by individual CTLA-4 binding aptamers. Individual aptamers from the pool of aptamers present after 9 rounds of selection were cloned and sequenced (M9-1, M9-2, etc.). Several members of the selected pool were represented more than once as indicated in the parentheses. The cloned aptamers were tested in vitro for inhibition of CTLA-4 function. The aptamers were used at two concentrations, 200 nM and 400 nM, corresponding to the estimated molar concentration of the anti CTLA-4 Ab binding sites used as positive control. Inhibition of CTLA-4 function is reflected in increased proliferation of T cells in the presence of anti-CTLA-4 Ab compared to isotype control Ab. Aptamers M9-8, M9-9, and M9-14, but not M9-15, inhibited CTLA-4 function in a dose responsive manner. FIG. 4B. Right: Inhibition of CTLA-4 function by M9-9 aptamer and a 35 nt long truncate derived by deletion from the 3′ end, del 60. Left: Computer simulated secondary structure of del 60 aptamer showing the proposed CTLA-4 binding site is shown.

FIG. 5. Inhibition of CTLA-4 function in vitro by the del 60 aptamers. In vitro functional assay for CTLA-4 inhibition was carried out as described in FIG. 4. Unconjugated del 60 and del 60/scram control aptamer (a scrambled sequence of del 60). In a further test of specificity, del 60 aptamer was preincubated with mCTLA-4/Fc or human IgG as indicated, then using protein G coupled to magnetic beads prior to use in the T cell proliferation reaction.

FIG. 6. Inhibition of tumor growth in mice treated with the CTLA-4 binding del 60 aptamers. C57BL/6 mice were injected with PBS, or implanted with B16/F10.9 melanoma tumors cells in the left flank and immunized with irradiated GM-CSF expressing B16/F10.9 tumor cells in the right flank on days 1, 3 and 6 following implantation. Antibody or aptamer was administered i.p as indicated on days 3 and 6 following tumor implantation. 5 mice were used in each treatment group. Individual (dots) and average (columns) tumor size is shown. Aptamers used were: Del 60 described above; del 55 a truncated aptamer derived from M9-14 (FIG. 4A) which also inhibited CTLA-4 function in vitro; M8G-28 aptamer generated in another selection experiment which did not inhibit CTLA-4 in an in vitro assay.

FIG. 7. TERT immunotherapy by CTLA-4 aptamers. C57BL/6 mice were injected with PBS, or implanted with B16/F10.9 melanoma tumors cells in the left flank and immunized with actin or TERT mRNA transfected DC 2, 9 and 16 days post tumor cell implantation. As indicated, Del 60 CTLA-4 binding aptamer or the control Del 60/SCRAM were administered to TERT immunized mice 3 and 6 days following each immunization. 5 mice were used in each treatment group. Individual (dots) and average (columns) tumor size is shown 21 days post tumor implantation.

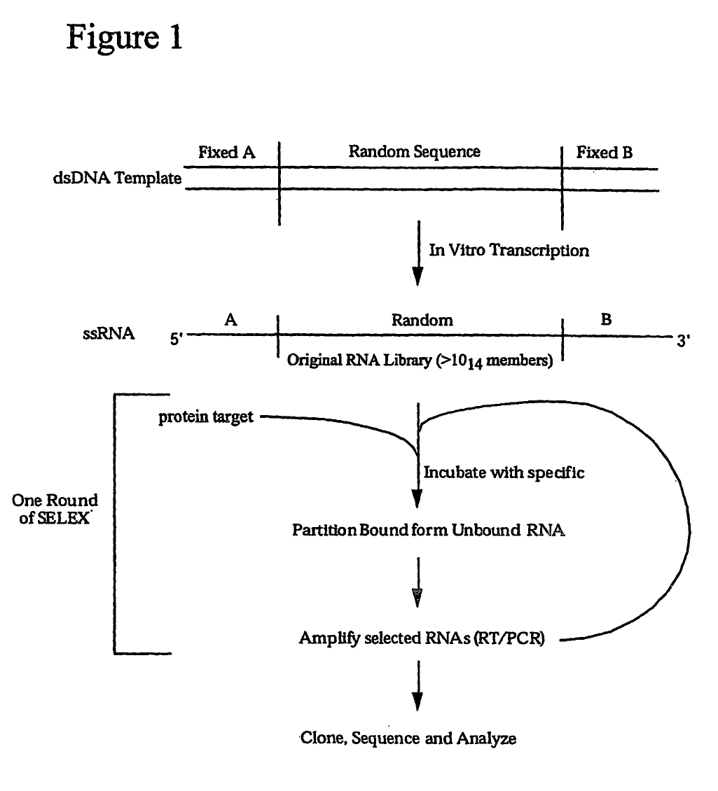

### DETAILED DESCRIPTION OF THE INVENTION

The present invention relates generally to a method of regulating immune function using nucleic acid ligands, or aptamers. In accordance with the invention, aptamers specific for targets including, but not limited to, CTLA-4, CD40, 4-1BB, OX40 and the TGFβ receptor, can be used to manipulate the immune system.

In a preferred embodiment, the invention relates to a method of cancer immunotherapy. In accordance with this method, an aptamer that inhibits the action of a negative regulator, for example, CTLA-4, can be used to potentiate a vaccine-induced antitumor immune response. Advantageously, the aptamer binds CTLA-4 but not CD28.

Aptamers suitable for use in the present invention can be isolated using, for example, the SELEX process (see, for example, U.S. Pat. Nos. 5,475,096, 5,270,163, 5,707,796, 5,763,177, 5,580,737, 5,567,588 and 6,171,795 and other such patents cited in WO 02/096926). Positive-negative selection schemes can be used to reduce the likelihood of an aptamer binding to a non-target molecule, for example, CD28 when CTLA-4 is the intended target. “Toggle SELEX” (White et al, Mol. Ther. 4(6):567-573 (2001)) can be used to isolate aptamers that cross-react with interspecies homologues of the same target (e.g., human CTLA-4 and its murine homolog). Such cross reactive aptamers can be used, for example, in assessing the function and toxicity of an aptamer in an in vivo animal model.

In isolating aptamers that target, for example, human CTLA-4, an RNA library that contains modified nucleotides can be used to yield aptamers that, for example, are resistant to nuclease degradation. The plasma stability of an aptamer can be increased by substitution of ribonucleotides with, for example, 2′-amino, 2′-fluoro, or 2′-O-alkyl nucleotides (Beigelman et al, J. Biol. Chem. 270(43):25702-25708 (1995), Pieken et al, Science 253(5017):314-317 (1991)). Modified-RNA oligonucleotides containing these substitutions can have in vitro half-lives in the 5 to 15 hour range. Furthermore, because 2′-amino or 2′-fluoro CTP and UTP can be readily incorporated into RNA by in vitro transcription, these backbone modifications can be introduced into the combinatorial library at the outset of the selection process (Aurup et al, Biochemistry 31(40):9636-9641 (1992), Jellinek et al, Biochemistry 34(36):11363-11372 (1995)). An aptamer can be protected from exonuclease degradation by capping its 3′ end (Beigelman et al, J. Biol. Chem. 270(43):25702-25708 (1995)). Resistance to endonuclease degradation can be further increased by additional substitution of ribose and deoxyribose nucleotides with modified nucleotides such as O-methyl modified nucleotides or various non-nucleotide linkers. The clearance rate of an aptamer can be rationally altered by increasing its effective molecular size, such as by the site-specific addition of various molecular weight polyethylene glycol (PEG) moieties or hydrophobic groups such as cholesterol or by attachment of the aptamer to the surface of a liposome (Tucker et al, J. Chromatogr. B. Biomed. Sci. Appl. 732(1):203-212 (1999), Willis et al, Bioconjug. Chem. 9(5):573-582 (1998)). An aptamer of the invention can thus be formulated in such a way as to have a half-life in vivo of a few minutes to several days. (See also modifications described in Lee and Sullenger, Nat. Biotechnol. 15(1):41-45 (1997), Lee and Sullenger, J. Exp. Med. 194(2):315-324 (1996), Gold, J. Biol. Chem. 270(23):13581-13584 (1995) and WO 02/096926).

Avidity and bioactivity can also be enhanced by generating mulimeric derivatives of aptamers (Altman et al, Science 274:94-96 (1996)).

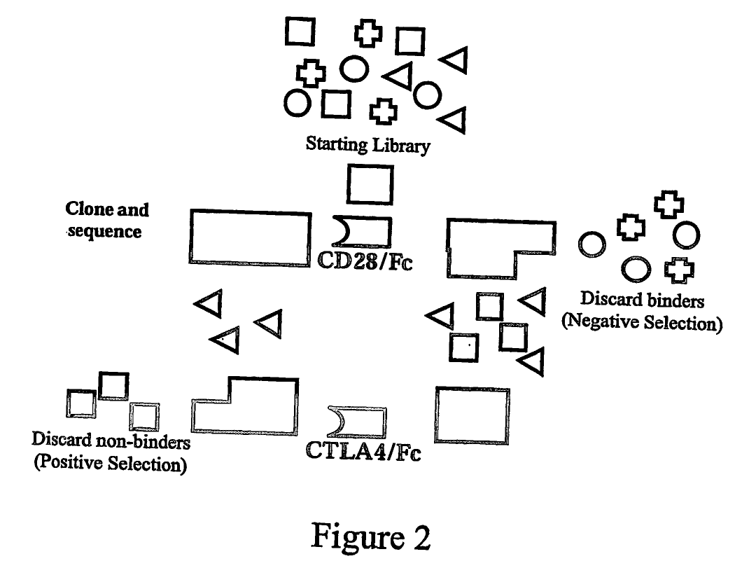

The following is provided for purposes of exemplification. An RNA library containing about a 20-40 nucleotide random sequence region flanked by fixed sequences can be generated, for example, by in vitro transcription of a synthetic DNA template (Rusconi et al, Thromb. Haemost. 84(5):841-848 (2000), Doudna et al, Proc. Natl. Acad. Sci. USA 92(6):2355-2359 (1995)). To identify and isolate RNA aptamers that recognize, for example, human CTLA-4, but that do not bind non-target molecules, for example, human CD28, a positive-negative SELEX process such as that described in FIG. 2 can be used. Randomized RNA libraries (˜10¹⁵ different molecules) can be screened for those RNAs that bind to the human CTLA-4 protein in the form, for example, of a CTLA-4/F_{c} fusion protein. To deplete those RNAs that also bind, for example, CD28, the RNA library can be preincubated with, for example, a human CD28/F_{c} fusion protein. RNAs that bind to the CD28 protein can be eliminated, for example, by precipitating the CD28/F_{c} fusion protein-RNA complexes with, for example, protein G-coated Sepharose beads. Such a process can also serve to preclear RNAs from the pool that bind, for example, to the protein G coated beads, F_{c} and CD28. The precleared RNA pool can then be incubated with, for example, the human CTLA-4/F_{c} fusion protein and RNAs that bind to this protein can be recovered, for example, by precipitation with protein G beads and elution, for example, via phenol extraction. Eluted RNAs can then be reverse transcribed, and the resulting cDNAs PCR amplified to generate DNA templates that can be in vitro transcribed to produce RNA for the next round of selection. Approximately 8 to 14 rounds of such selection can be used to yield RNA molecules that bind to the human CTLA-4/F_{c} fusion protein with high affinity and specificity. The sequences of these RNA molecules can be determined and their affinities for human CTLA-4, CD28 and human F_{c} determined, for example, by Biacore or nitrocellulose filter binding methods (Rusconi et al, Thromb. Haemost. 84(5):841-848 (2000)).

RNA aptamer-protein equilibrium dissociation constants (K_{d}'s) can be determined using, for example, a double filter, nitrocellulose-filter binding method (Conrad et al, J. Biol. Chem. 269(51):32051-32054 (1994), Rusconi et al, Thromb. Haemost. 84(5):841-848 (2000)). Briefly, and merely for purposes of exemplification, ³²P-end-labeled RNA aptamers (<0.1 nM) can be incubated with the individual proteins at a range of concentrations. The RNA-protein complexes can be separated from free RNA, for example, by passing the mixture through a nitrocellulose filter. Bound and free RNA and can be quantitated, for example, by phosphorimager analysis and the data fitted to yield the K_{d}s for the RNA aptamer-protein interaction. Advantageously, aptamers are isolated that bind target (e.g., human CTLA-4) with a K_{d} in the high picomolar to low nanomolar range but that do not bind non-target molecules (e.g., human CD28 or F_{c}) any tighter than the original RNA library binds such molecules.

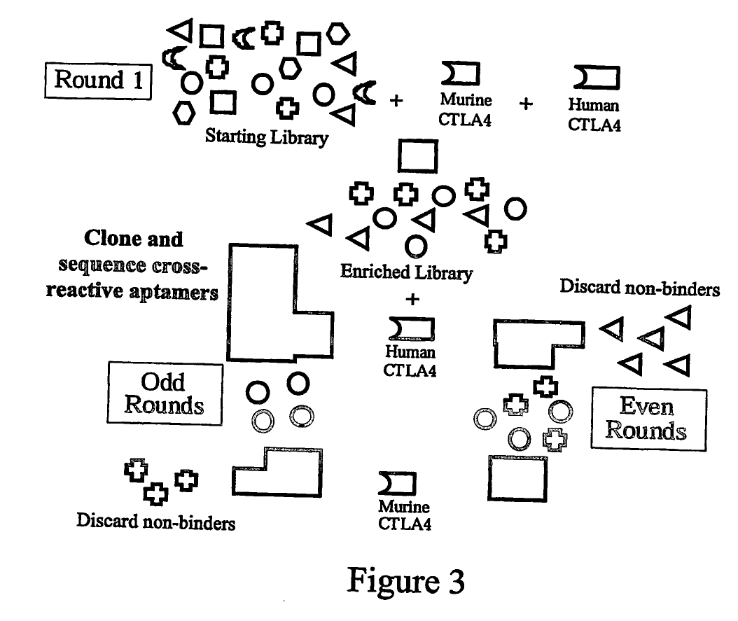

As indicated above, “toggle SELEX” can be used to identify aptamers that recognize conserved epitopes on interspecies homologues (e.g., human and murine) of a target, for example, CTLA-4 (White et al, Mol. Ther. 4(6):567-573 (2001)). To isolate such cross-reactive aptamers, alternate rounds of selection can be performed with, for example, the human and murine CTLA-4/F_{c} proteins as shown in FIG. 3. In the first round of in vitro selection, the starting library of RNAs can be incubated with, for example, both human and murine CTLA-4/F_{c}. RNAs that bind to either protein can be recovered, for example, by precipitation with protein G-beads and amplified for the next round of selection. In the second round of selection, the enriched library can be incubated with, for example, human CTLA-4/F_{c} alone and bound RNAs recovered to generate a library of RNAs that have been further enriched for members that bind surfaces on human CTLA-4/F_{c}. In round 3 of selection, this human CTLA-4 enriched library can be incubated with murine CTLA-4/F_{c} and the subset of RNAs that bind the murine protein recovered. RNAs that do not bind the murine protein can be discarded. In this example, the resulting RNA library is enriched for RNAs that bind structural motifs that are conserved between the human and murine CTLA-4 proteins. Approximately, 8-14 rounds of toggle SELEX can be expected to yield RNA aptamers that bind to both human and murine CTLA-4 proteins. During the toggle SELEX process, positive-negative selection can be performed as described above. Aptamers can be isolated using this approach that bind to both human and murine CTLA-4 with K_{d}s in the low nanomolar to high picomolar range but that do not bind human and murine CD28 any tighter than the original RNA library. It will be appreciated that the foregoing approach is applicable to other target proteins.

Subsequent truncation studies can be used to identify aptamers less than, for example, about 50 nucleotides in length that bind target, e.g., CTLA-4. Mutagenesis studies can be used to generate control aptamer(s) that do not bind target (e.g., CTLA-4) but that are very similar in sequence to the wild type aptamer(s). Such mutant aptamers can serve as negative control(s) in in vitro and in vivo studies.

Truncation and mutagenesis studies can be carried out using standard techniques. However, the following is provided for purposes of exemplification. To develop truncate and mutant aptamers, aptamers isolated using approaches such as those described above can be grouped into families utilizing, for example, RNA sequence alignment (Davis et al, Methods Enzymol. 267:302-314 (1996)) and RNA folding algorithms (Mathews et al, J. Mol. Biol. 288(5):911-940 (1999)) as previously described (Rusconi et al, Thromb. Haemost. 84(5):841-848 (2000)). Once grouped, covariation analysis can be employed to develop an initial secondary structure model of how the aptamers fold in each family. This model can be tested by making specific mutations that can be predicted to disrupt the folding of the aptamer as well as compensatory mutations that can be expected to restore the structure. In addition, regions of the aptamer not important for folding in the working model can be deleted and the ability of all of these aptamer variants to bind target (e.g., CTLA-4) assessed as described above. In addition, aptamer derivatives containing one or only a few point mutations in highly conserved sequences within an aptamer family can be generated and tested to identify nucleotides critical for aptamer binding to target.

Aptamers selected using approaches such as those described above can be further selected based on their ability to compete with a known ligand for binding to the target. In the case of CTLA-4, aptamers can be screened based on their ability to compete with, for example, B7 (White et al, Mol. Ther. 4(6):567-573 (2001), Rusconi et al, Thromb. Haemost. 84(5):841-848 (2000)). In brief, and merely by way of example, trace amounts of ³²P-labelled aptamer can be incubated with CTLA-4 under conditions that allow for approximately one-half of the aptamer to bind the protein. In addition, increasing amounts of B7-1 protein can be added to determine if B7 can compete with the aptamer for CTLA-4 binding. The binding reactions can then be passed through the nitrocellulose/nylon two filter system to separate aptamer that is bound to CTLA-4 from unbound aptamer. Radioactivity on the filters can be quantitated using, for example, phosphorimager analysis and the data used to quantitate the kᵢ for B7 competition.

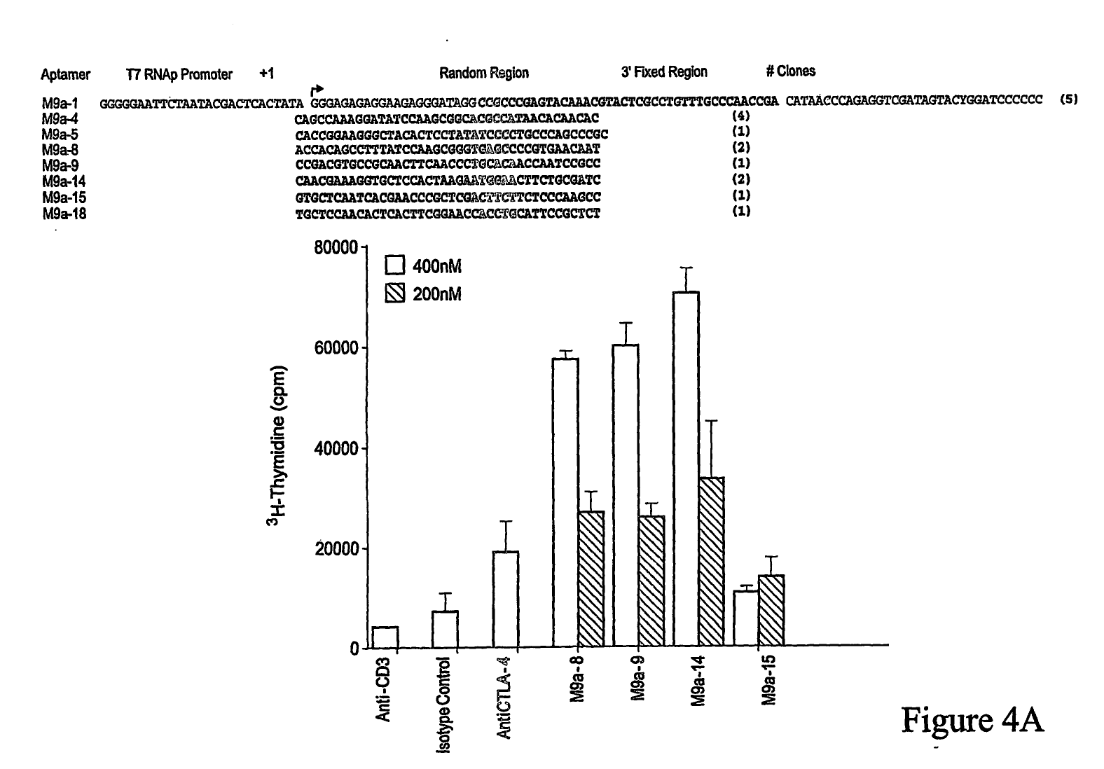

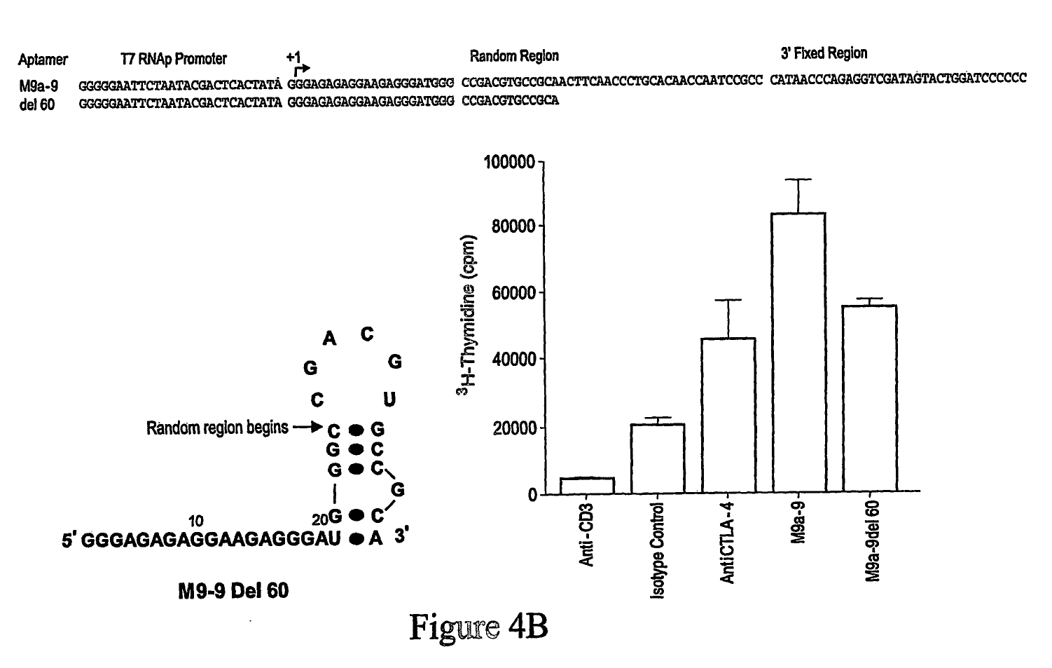

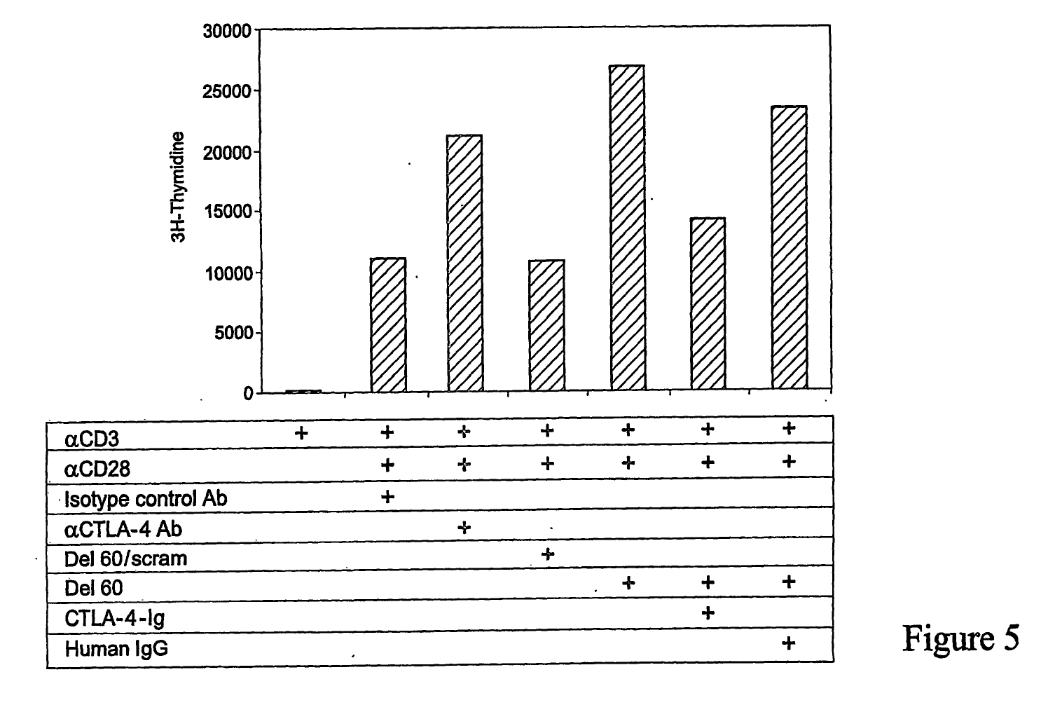

Aptamers selected using approaches such as those described above can be tested for activity using in vitro assays. For example, aptamers selected for binding to murine and/or human CTLA-4 can be tested for their ability to block the function of CTLA-4 in vitro. Blocking CTLA-4 function can be measured, for example, in an in vitro proliferation assay. The readout can be, for example, an enhancement of T cell proliferation under conditions of suboptimal polyclonal activation with α-CD3 and α-CD28 in the presence of a CTLA-4 inhibitor, such as α-CTLA-4 (Krummel and Allison, J. Exp. Med. 182(2):459-465 (1995), Walunas et al, Immunity 1(5):405-413 (1994)) or CTLA-4 binding aptamers (FIGS. 4 and 5). Because T cell proliferation enhancement due to CTLA-4 blockade is detectable under suboptimal conditions, the concentrations of human α-CD3 and α-CD28 as well as α-CTLA-4 required to detect enhanced proliferation of human T cells can be determined empirically. To determine incubation time, the cells can be harvested over a time course, for example, pulsing with ³H-thymidine for 14-18 hours prior to harvest. Like murine T cells, hCTLA-4 expression peaks at 2-3 days, however, on human T cells, expression remains high for at least 5 days potentially making increased incubation time advantageous (Linsley et al, J. Exp. Med. 176(6):1595-1604 (1992), Wang et al, Scand. J. Immunol. 54(5):453-458 (2001)). In mice, proliferation enhancement mediated by α-CTLA-4 occurs by upregulating CTLA-4 expression which is not detectable on resting T cells. However, resting human T cells have detectable CTLA-4 expression that is upregulated upon activation (Wang et al, Scand. J. Immunol. 54(5):453-458 (2001), Lindsten et al, J. Immunol. 151(7):3489-3499 (1993)). To account for this, α-CTLA-4 can be added at varying time points during culture. Culture to culture variations in the proportion of dividing cells can be assessed and minimized using replicates for each condition.

An alternative method to upregulate human CTLA-4 expression is incubation with IL-2, which functions in a dose dependent manner on human T cells (Wang et al, Scand. J. Immunol. 54(5):453-458 (2001)). Concentration of IL-2 and length of incubation can be empirically tested to identify conditions for proliferation enhancement with α-CTLA-4 antibody. Prior to addition of α-CTLA-4, cultures can be washed to remove the IL-2 if presence of this cytokine diminishes the effect of α-CTLA-4 on T cell proliferation.

Serial dilutions of an aptamer (e.g., an aptamer that targets CTLA-4) can be tested over a range of concentrations above and below that which gives the equivalent number of binding sites as the optimal concentration of, in the case of CTLA-4, α-CTLA-4 antibody (for example). To confirm that enhancement of T cell proliferation is due to inhibition of CTLA-4 function, two controls can be used: a) control oligonucleotides (ODNs) with similar base composition (scrambled ODNs) that do not bind CTLA-4 can be used as a negative control, and b) aptamer candidates can be preincubated with, for example, hCTLA-4/F_{c} or control Ig to remove the aptamer prior to addition to the T cell culture, as shown in FIG. 5. If enhancement is due to CTLA-4:aptamer interaction, rather than F_{c}:aptamer interaction, the CTLA-4 mediated enhancement of proliferation is ablated upon preclearing with hCTLA-4/F_{c}, but not control Ig, as seen in the case of the murine aptamers (FIG. 5). Those aptamers that consistently enhance proliferation to a comparable level at a comparable concentration or less than, for example, α-CTLA-4 are preferred candidates for further testing. Aptamers that block CTLA-4 at the lowest concentrations can be further truncated and retested for CTLA-4 binding and CTLA-4 blockade of function.

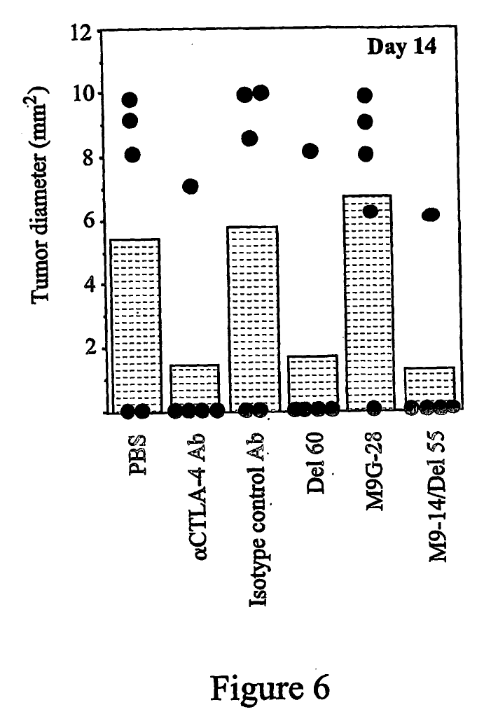

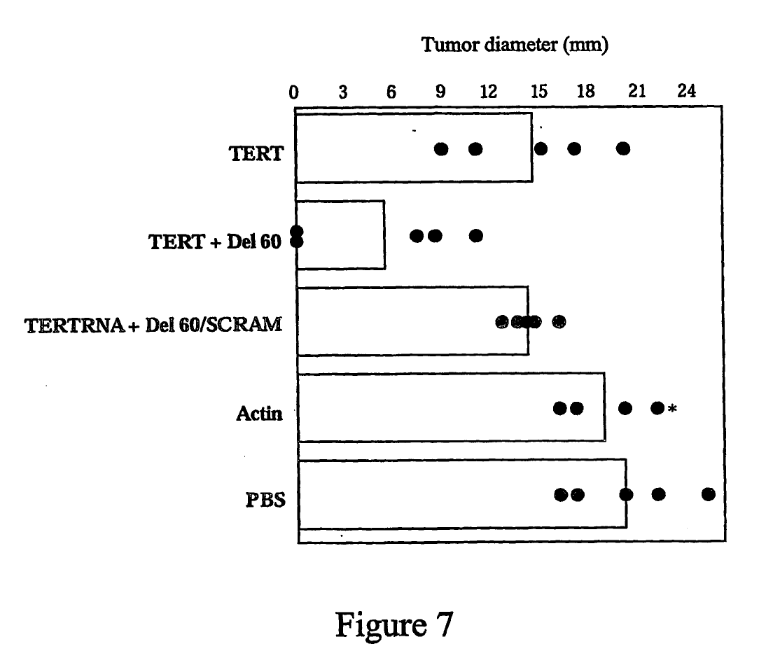

The B16/F10.9 melanoma tumor model can be used to assess the toxicity of aptamers and their derivatives and to test the ability of aptamers to prevent or delay tumor growth in female C57BL/6 mice (Porgador et al, J. Immunogenet. 16(4)-5):291-303 (1989)) (see FIGS. 6 and 7). Mice can be immunized with TERT mRNA transfected DC and the ability of aptamers to enhance antitumor immunity can be determined as described, for example, in FIG. 7. The stringency of this therapeutic model can be controlled via the dose of tumor cells implanted or the interval between tumor cell implantation and start of immunotherapy/aptamer administration. This permits the evaluation of increasingly effective aptamers and their derivatives.

Mice treated with selected aptamers exhibiting potent antitumor responses can be analyzed for signs of autoimmunity. To screen for dysregulated lymphoproliferation, immunized mice can be periodically sacrificed and subjected to detailed pathological analysis and blood immunohistochemistry.

Pharmaceutically useful compositions comprising aptamers of the invention can be formulated using art recognized techniques with a pharmaceutically acceptable carrier, diluent or excipient. Examples of such carriers and methods of formulation can be found in Remington's Pharmaceutical Sciences. Aptamers can be formulated, for example, as solutions, creams, gels, ointments or sprays. The aptamers can be present in dosage unit forms, such as pills, capsules, tablets or suppositories. When appropriate, the compositions can be sterile. The compositions can comprise more than one aptamer of the invention.

Modes of administration of aptamers of the invention, or composition comprising same, can vary with the aptamer, the patient and the effect sought. Examples of such modes include parenteral, intravenous, intradermal, intrathecal, intramuscular, subcutaneous, topical, transdermal patch, via rectal, vaginal or urethral suppository, peritoneal, percutaneous, nasal spray, surgical implant, internal surgical paint, infusion pump or via catheter. The aptamer, or composition comprising same, can be administered in a slow release formulation such as an implant, bolus, microparticle, microsphere, nanoparticle or nanosphere. For standard information on pharmaceutical formulations, see Ansel, et al., Pharmaceutical Dosage Forms and Drug Delivery Systems, Sixth Edition, Williams & Wilkins (1995).

Aptamers of the invention can be administered in a manner compatible with the dosage formulation, and in a therapeutically effective amount. The quantity to be administered can vary with the aptamer, the subject to be treated, capacity of the subject's system to utilize the active ingredient, and degree of therapeutic effect desired. Optimum amounts of aptamer required to be administered can readily be determined by one skilled in the art. Generally, the compositions can be administered in dosages adjusted for body weight, e.g., dosages ranging from about 1 μg/kg body weight to about 100 mg/kg body weight, preferably, 1 mg/kg body weight to 50 mg/kg body weight.

Aptamers of the invention, particularly CTLA-4 targeted aptamers, serve as a useful adjunct to Ag-specific immunotherapy to potentiate the vaccine generated antitumor responses in both human and non-human mammals.

Certain aspects of the invention can be described in greater detail in the non-limiting Examples that follows.

### EXAMPLE 1

Isolation of nuclease-resistant RNA aptamers that inhibit the function of CTLA-4 was accomplished using the SELEX protocol to isolate CTLA-4 binding aptamers (Lee et al, New Biol. 4(1):66-74 (1992), Ellington and Szostak, Nature 346(6287):818-822 (1990)). Briefly, a library of >10¹⁴ unique RNA molecules was generated whereby each molecule is comprised of a 40 nt long random region flanked by constant sequences used to amplify the selected RNA species for successive rounds of selection. To increase RNase-resistance, 2′-fluoro-modified pyrimidines were incorporated into the molecules during transcription. The RNA library was incubated with murine CTLA-4/human Fc fusion protein (mCTLA-4/Fc), bound RNA was partitioned from non bound RNA by nitrocellulose filter binding and subjected to a subsequent round of selection. After every two rounds, the affinity of RNA to mCTLA-4/F_{c} was checked using the filter binding assay to monitor progress of the selection; increased affinity indicates the selection is advancing. Specificity was tracked by intermittent measurements for CD28 and human IgG (huIgG) binding affinities. The selection was carried out for 9 rounds at which point no further increase in affinity was seen. The pool of CTLA-4 binding aptamers did not exhibit binding for CD28 despite the considerable homology between these two molecules (Chambers et al, Immunity 7(6):885-895 (1997), Alegre et al, Nature Reviews Immunology 1:220-228 (2001), Salomon and Bluestone, Annu. Rev. Immunol. 19:225-252 (2001)).

Cloning and sequencing of the amplification products from round 9 revealed limited sequence diversity, with 8 unique sequences represented multiple times (FIG. 4A), indicating that the selection was nearly at an endpoint. Members representing each sequence were tested in an in vitro assay for CTLA-4 inhibition. In this assay, purified T cells are suboptimally stimulated to proliferate by incubation with anti-CD3 and anti-CD28 Ab as previously described (Krummel and Allison, J. Exp. Med. 182(2):459-465 (1995), Walunas et al, Immunity 1(5):405-413 (1994)). Consistent with the function of CTLA-4 to attenuate T cell proliferation, incubation with anti-CTLA-4 Ab, but not with an isotype control Ab, resulted in an enhancement of T cell proliferation. Several RNA species inhibited CTLA-4 function comparably or better than anti-CTLA-4 antibody (FIG. 4A: M9-8, M9-9 and M9-14), whereas other RNA species did not inhibit CTLA-4 function despite the fact that they bound to CTLA-4 (FIG. 4A: M9-15).

Aptamer M9-9 was chosen for further study on the basis of being consistently the most potent inhibitor of CTLA-4 function as shown in FIG. 4A. To facilitate the in vivo analysis of the CTLA-4 binding aptamers, deletion derivatives of the M9-9 aptamer were generated and tested for CTLA-4 binding and the ability to inhibit CTLA-4-4 function in vitro. The smallest functional M9-9 aptamer which bound to mCTLA-4/F_{c} and inhibited CTLA-4 function was the 35 nt long truncate designated Del 60 (FIG. 4B). A computer simulated secondary structure of Del 60 suggests that a stem-loop structure constitutes the binding site for CTLA-4 (FIG. 4B).

The specificity of inhibition of CTLA-4 function by the Del 60 aptamer is shown in FIG. 5. First, Del 60, but not the control aptamer, enhances T cell proliferation under limiting conditions. Second, preincubation of the Del 60 aptamer with mCTLA-4/Fc, but not huIgG, prior to addition to the T cell culture abrogated the enhancing effect of the aptamer, showing that Del 60 mediates its effect by binding to the CTLA-4 and not the Fc portion of mCTLA-4/Fc.

Murine studies have shown that rejection of tumors can be achieved if antibody-mediated CTLA-4 blockade is used in combination with vaccination under conditions that neither treatment is effective alone (Hurwitz et al, Proc. Natl. Acad. Sci. USA 95(17):10067-10071 (1998), Hurwitz et al, Cancer Res. 60(9):2444-2448 (2000), van Elsas et al, J. Exp. Med. 190(3):355-366 (1999)). The ability of the CTLA-4 binding aptamers to impact on tumor growth was first tested in the poorly immunogenic B16/F10.9 melanoma model (Porgador et al, J. Immunogenet. 16(4)-5):291-303 (1989)) used in the previous studies (Hurwitz et al, Proc. Natl. Acad. Sci. USA 95(17):10067-10071 (1998), Hurwitz et al, Cancer Res. 60(9):2444-2448 (2000), van Elsas et al, J. Exp. Med. 190(3):355-366 (1999)). In the experiment shown in FIG. 6, mice were implanted with B16/F10.9 tumor cells and either mock immunized with PBS or immunized with irradiated GM-CSF secreting B16/F10.9 (F10.9-GM) tumor cells on day 1, 3 and 6 following implantation. On day 3 and 6 following tumor implantation, the F10.9-GM immunized group but not the mock immunized PBS group, were injected i.p. with either antibody or aptamer. In this experiment, immunization with irradiated F10.9-GM cells had no discernible effect on tumor growth (compare PBS group which did not receive the F10.9-GM cell vaccine to the immunized group treated with isotype control Ab). Conceivably, the weak immune response elicited by immunizing the mice with irradiated GM-CSF secreting B16/F10.9 tumor cells was not sufficient to affect tumor growth in a measurable way. As previously reported, immunized mice treated with anti-CTLA-4 Ab, but not isotype control Ab, exhibited a significant delay in tumor growth (van Elsas, 1999 #23; Hurwitz, 1998 #21; Hurwitz, 2000 #22). Two aptamers which inhibited CTLA-4 function in vitro, Del 60 and M9-14 del 55, a truncated form of M9-14 which also inhibits CTLA-4 function in vitro, but not M8G-28 which binds CTLA-4 but does not inhibit CTLA-4 function in vitro, inhibited tumor growth to a comparable extent seen in mice treated with anti-CTLA-4 Ab. This experiment therefore provides evidence that the CTLA-4 binding aptamers are biologically active in vivo.

It has been shown previously that immunization of mice against the protein subunit of murine telomerase, telomerase reverse transcriptase (TERT), using TERT mRNA transfected syngeneic bone marrow derived dendritic cells (DC), engenders protective antitumor immunity (Nair et al, Nat. Med. 6(9):1011-1017 (2000)). The attractive feature of immunotherapy against TERT is that it is overexpressed in most tumors (>80%) (Kim et al, Science 266(5193):2011-2015 (1994), Shay and Bacchetti, Eur. J. Cancer 33(5):787-791 (1997)) and hence represent a common target for immunotherapy. Yet, not surprisingly since TERT is a normal gene product it is a weak antigen, namely the antitumor response stimulated by immunization against TERT was modest (Nair et al, Nat. Med. 6(9):1011-1017 (2000)). It was speculated that in order to enhance the therapeutic impact of immunization it will be useful to target additional tumor-expressed antigens and/or to combine anti-TERT immunization with other treatments. Here, it was tested whether treatment of mice with CTLA4 binding aptamers would enhance the therapeutic benefit of immunization against TERT. As shown in FIG. 7, treatment of tumor bearing animals with TERT mRNA transfected DC had a very modest tumor inhibitory effect, consistent with previous observations (Nair et al, Nat. Med. 6(9):1011-1017 (2000)). However, when the mice were also treated with a CTLA-4 binding aptamer (Del 60) which was also shown to inhibit CTLA4 function in vitro (FIGS. 5 & 6), but not with a non functional non CTLA-4 binding control aptamer (Del 60/SCRAM), tumor inhibition was significantly enhanced. This observation provides additional evidence to support the conclusion that the CTLA-4 binding aptamers isolated by the SELEX procedure exhibit biological activity.

### EXAMPLE 2

Aptamers that bind and antagonize human CTLA-4 can be optimized for activity as inhibitors by modifying them to have increased in vivo stability, increased circulating half-lives and increased avidity for human CTLA-4. In each case, the fact that aptamers are synthetic compounds that can be modified by post synthetic chemical methods to yield derivatives with desired properties can be exploited.

Enhancing stability. To render the 2′-flouro-pyrimidine containing CTLA-4 aptamers even more resistant to nuclease-degradation in vivo, they can be further modified by replacing as many 2′-hydroxy (2′OH) purines as possible (without significant loss in CTLA-4 binding affinity-ensuring by functional analysis) with modified purine nucleotides that contain 2′-O-methyl (2′Ome) on their sugars. Such substitutions have been previously shown to further enhance the nuclease stability of aptamers in vivo (for review see Hicke et al, J. Clin. Invest. 106:923 (2000)). Unfortunately, 2′Ome purines are difficult to incorporate into RNA during SELEX because T7 RNA polymerase does not utilize them well during in vitro transcription. Thus such modifications can be incorporated into the CTLA-4 aptamers after identification, truncation and validation. Synthesis of 2′-Ome-containing oligonucleotides is now standard practice and such compounds are commercially available. It is expected that the majority of the 2′OH purines can be replaced by 2′Ome to yield modified CTLA-4 specific aptamers that are highly resistant to nuclease degradation in vivo (Hicke et al, J. Clin. Invest. 106:923 (2000)). To determine which purines can be substituted without loss of CTLA-4 binding, the working secondary structural model of the aptamer can be exploited. First the aptamer can be divided into structural domains (e.g., various stems and loops). Derivatives of the aptamer can then be synthesized that contain each purine residue in a given domain modified to contain a 2′Ome for each domain. These modified aptamers can then be tested in binding studies to determine if such substitution impacts aptamer-CTLA-4 binding. If a particular domain(s) does not tolerate total 2′Ome substitution (result in greatly reduced binding), which nucleotide(s) within that domain cannot be 2′Ome substituted can be determined by generating and analyzing derivatives with single 2′Ome substitutions. In this manner those purines that can be modified with 2′Ome and those that cannot can be readily identified. Finally, a CTLA-4 aptamer that contains the maximum number of allowable 2′Omes can be generated and tested in biochemical, cell and in vivo studies.

Enhancing bioavailability. To enhance the bioavailability of aptamers in vivo, it has been shown that addition of either a cholesterol moiety or a 40 kDa polyethylene glycol (PEG) to the end of an aptamer can significantly improve the circulating half-life of these molecules in animal studies (Tucker et al, J. Chrom. B. Biomed. Sci. Appl. 732:203 (1999), Watson et al, Antisense Nucleic Acid Drug Dev. 10:63 (2000)). Aptamers without such post-synthetic modifications have circulating half-lives in the 10 minute range because they are cleared quickly by the kidney (Tucker et al, J. Chrom. B. Biomed. Sci. Appl. 732:203 (1999), Watson et al, Antisense Nucleic Acid Drug Dev. 10:63 (2000)). By contrast, aptamers containing cholesterol or PEG circulate with a half-life in the 4-12 hour range following IV administration (Tucker et al, J. Chrom. B. Biomed. Sci. Appl. 732:203 (1999), Watson et al, Antisense Nucleic Acid Drug Dev. 10:63 (2000)). Enhancing the circulating half-life of these compounds can translate into improved aptamer efficacy in vivo by providing a larger window of opportunity for the aptamer to bind to CTLA-4. To enhance the circulating half-life of the CTLA-4 aptamers in vivo, a cholesterol or a 40 kDa PEG moiety can be appended to an aptamer. These moieties can be attached to the 5′-end of the aptamer through a 6 carbon atom linker. The resulting aptamer derivatives can be assayed for their ability to bind CTLA-4 and inhibit its function. It has been demonstrated that attachment of a cholesterol and PEG moiety in this manner to an aptamer specific for coagulation factor IXa did not significantly impact on the ability of the aptamer to bind and inhibit factor IXa activity. Addition of a cholesterol to this aptamer (Reg1) significantly enhanced the circulating half-life of the aptamer following intravenous administration to swine. The unmodified aptamer has a circulating half-life of approximately 10 minutes whereas the cholesterol modified aptamer circulates with a half life of greater than 3 hours. It has been demonstrated that PEG-modified aptamers can circulate with half lives in the 12 hour range following IV administration (Tucker et al, J. Chrom. B. Biomed. Sci. Appl. 732:203 (1999), Watson et al, Antisense Nucleic Acid Drug Dev. 10:63 (2000)). If addition of the cholesterol or 40 kDa PEG significantly reduces binding of an aptamer to CTLA-4, then other length linkers can be examined for their attachment to the aptamer as can attachment of the cholesterol and PEG at the 3′ end of the aptamer. Chemistry for such 3′end attachment is less well developed than 5′ end attachment, thus, modification of the 5′ end is preferred. Finally, CTLA-4 aptamer derivatives that tolerate cholesterol or PEG addition can be screened in cell based and in vivo assays for activity.

Mulimeric forms of tetramers can be generated to enhance their avidity to CTLA-4 and bioactivity in vivo (Altman et al, Science 274:94-96 (1996)). As an example, three strategies are described below:

i) Bivalent aptamer synthesis. Ringquist and Parma (Cytochemistry 33:394 (1998)) have described the synthesis of bivalent versions of an aptamer against L-selectin using solid phase phosphoramidite coupling chemistry initiated from a branched 3′-3′ linked CPG support (Glen Research, Sterling, Va.). This strategy can be used to generate bivalent versions of aptamers in which the 3′ ends of the aptamer units are joined via the symmetric linker. This strategy allows for easy alteration of the distance between aptamer units by inclusion of variable atom-length spacers (eg., 3, 6, 9 or 18 atom spacers) by incorporation of the spacer between the CPG and the 3′ residue of the aptamer using standard phophoramidite linkers and coupling chemistry. This method is validated and can enable the controlled generation of bivalent aptamers. The limitations of this method are that only 3′ linked bivalent aptamers can be synthesized, and overall yields may be low due to the number of coupling steps.

ii) Tri- and tetravalent aptamer synthesis. Dendrimer phophoramidites (Shchepinov et al, Nucleic Acids Res. 25:4447 (1997)) (Glen Research, Sterling, Va.) can be employed to generate tri- and tetravalent formulation. A dendrimer phosphoramidite synthon is essentially a building block that can be used to increase the valency of a monomeric oligonucleotide, as addition of this synthon to an oligonucleotide creates, depending on the synthon used, two to three sites for additional oligonucleotide synthesis or attachment. This synthesis strategy has been used to make multivalent PCR primers, hybridization probes, etc. (Shchepinov et al, Nucleic Acids Res. 25:4447 (1997), and references therein), and is readily transferable to synthesis of multivalent aptamers. Briefly, aptamers synthesized from an inverted deoxythymidine CPG as currently done can be coupled to a symmetric doubler or trebler dendrimer phosphoramidite onto the 5′ residue to create 2 or 3 additional sites, respectively, for aptamer attachment. Additional units can then be added by step-wise synthesis of the aptamer from the dendrimer to create tri or tetravalent aptamers depending on the dendrimer used. Alternatively, a dA-5′-CE phosphoramidite can be coupled to the dendrimer, and then additional units can be attached to the dendrimer by coupling of the 5′ end of a previously synthesized aptamer unit (still containing its 5′ DMT) via a 5′-5′ linkage to this site to create tri and tetravalent aptamers joined at their respective 5′ ends. This latter strategy has the advantage of coupling fully synthesized aptamers to the dendrimer, and can, therefore, result in a cleaner product at higher yields. As above, spacing between individual aptamer units can be adjusted by inclusion of variable atom spacers between the aptamer units and dendrimer attachment sites. Of these schemes, the latter is more likely to produce a cleaner product at higher yields, as purified full-length aptamers are used to assemble the final multivalent product.

iii) Bi- to pentavalent aptamer synthesis. Beier and Hoheisel (Nucleic Acids Res. 27:1970 (1999)) have described the synthesis of a flexible polyamine linker for the generation of multivalent nucleic acid probes. Essentially, this chemistry can be used to generate, in a controlled fashion, a flexible linker system containing 2 through N attachment sites (separated by defined linker distances) for oligonucleotides via simple conjugation chemistry. This approach can be used to generate bi to pentavalent formulations. Briefly, linkers with 2, 3, 4 or 5 primary amine attachment sites can be synthesized as described (Beier and Hoheisel, Nucleic Acids Res. 27:1970 (1999)). The linker can then be loaded with aptamer units by conjugation of the 5′hydroxyl of the previously synthesized aptamer to the amino group of the linker using disuccinimidylcarbonate or disuccinimidyloxalate as the activating agent. As with the above methods, the distance between the aptamer units can be varied, in this case by either increasing the spacing between the amino groups on the linker or adding variable length spacers to the 5′ends of the aptamer during synthesis. The resulting aptamers can be joined at their 5′ ends. Again, as full-length aptamers are used to assemble the final multivalent product, this method can produce a clean final product.

Multi-Valent Aptamer Characterization. Independent of the method used to synthesize multivalent aptameric derivatives, characterization of the number of aptamer units and the change in affinity engendered by these units can be critical to understanding the underlying mechanisms responsible for the increased potency of the polyvalent aptamers as seen in preliminary studies. The valency of the aptamer formulations can be readily confirmed by determination of the molecular weight of the aptamer formulation by Maldi-TOF mass spectrometry, and is a standard quality control step in aptamer synthesis at Transgenomic. The affinity of multivalent formulations can be determined by flow cytometry, and can employ fluorescently labeled versions of the aptamers and bead immobilized CTLA-4 (Ringquist et al, Cytometry 33:394 (1998), Davis et al Nucleic Acids Res. 26:3915 (1998)). This assay format allows for measurement of dissociation constants by titration and competition, as well as determination of the association and dissociation rates of the various aptamer formulations. Increasing the valency can lead to an increase in the affinity of the aptamer for bead-immobilized (and cell surface) CTLA-4, as well as increased residence time. While the activity of each aptamer unit of the multivalent aptamer formulations cannot be directly assessed, there is a strong theoretical basis from which expected increases in ligand affinity as a function of ligand valency can be predicted (Crothers et al, Immunochemistry 9:341 (1972), Kaufman et al, Cancer Res. 52:4157 (1992)). Comparison of the measured affinity of these formulations for bead-immobilized CTLA-4 with expected increases in affinity based upon ligand valency make it possible to estimate how many of the aptamer units have retained substantial CTLA-4 binding activity, and can be used to determine which formulation should be further tested in activity assays in vitro and in vivo.

All documents cited above are hereby incorporated in their entirety by reference.

What is claimed is:

1. A method of regulating immune function in a mammal comprising administering to said mammal an amount of an aptamer that binds CTLA-4, CD40, 4-1BB, OX40 or TGFβ receptor sufficient to effect said regulation.

2. The method according to claim 1 wherein said mammal is a cancer patient.

3. The method according to claim 2 wherein said patient is a human.

4. The method according to claim 2 wherein said patient is undergoing antigen specific immunotherapy.

5. The method according to claim 4 wherein administration of said aptamer potentiates said immunotherapy.

6. The method according to claim 1 wherein said aptamer binds CTLA-4.

7. The method according to claim 6 wherein said aptamer does not bind CD28.

8. The method according to claim 6 wherein said aptamer is specific for a conserved epitope of interspecies homologues of CTLA-4.

9. The method according to claim 6 wherein said aptamer is Del 60.

10. The method according to claim 1 wherein said aptamer is resistant to nuclease degradation.

11. The method according to claim 10 wherein said aptamer comprises a modified nucleotide selected from the group consisting of a 2′-amino, 2′-fluoro or 2′-O-alkyl nucleotide.

12. The method according to claim 11 wherein said aptamer bears a 3′ cap.

13. The method according to claim 1 wherein said aptamer comprises a polyethylene glycol moiety.

14. The method according to claim 1 wherein said aptamer is bound to a liposome.

15. The method according to claim 1 wherein said aptamer is present as a multimer.

16. A method of identifying an a CTLA-4 aptamer comprising:
i) preparing a candidate mixture of nucleic acids,
ii) contacting said mixture with CTLA-4, wherein nucleic acids present in said mixture having an increased affinity for CTLA-4 relative to the candidate mixture can be partitioned from the remainder of the candidate mixture, and
iii) partitioning said nucleic acids having an increased affinity for CTLA-4 from the remainder of said candidate mixture.

17. The method according to claim 16 further comprising, between steps (i) and (ii), contacting said candidate mixture with CD28 and partitioning out of said candidate mixture nucleic acids having an increased affinity for CD28 relative to the candidate mixture.

18. A method of identifying an aptamer that inhibits CTLA-4 function comprising assessing T cell proliferation in the presence and absence of said aptamer, enhancement of T cell proliferation in the presence of said aptamer indicating inhibition of CTLA-4 function.

19. An aptamer that binds CTLA-4.

20. The aptamer according to claim 19 wherein said aptamer is Del 60 or M9-14 del 55.

21. A composition comprising the aptamer of claim 19 and a carrier.
